(19) United States
(12) Patent Application Publication
(10) Pub. No.: US 2008/0057118 A1
(43) Pub. Date: Mar. 6, 2008

(54) DIVALPROEX PHARMACEUTICAL COMPOSITIONS

(76) Inventors: Alagumurugan Alagarsamy, Tuticorin (IN); Sudeep Kumar Agrawal, Raipur (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/847,457

(22) Filed: Aug. 30, 2007

Related U.S. Application Data
(60) Provisional application No. 60/870,005, filed on Dec. 14, 2006.

(30) Foreign Application Priority Data
Sep. 1, 2006 (IN) 1594/CHE/2006

(51) Int. Cl. A61K 9/52; A61K 31/19; A61K 9/16; A61P 25/00; A61K 9/26

(52) U.S. Cl. 424/457; 424/470; 424/490; 514/557

(57) ABSTRACT

Pharmaceutical compositions comprising a valproic acid drug compound, providing modified release of the drug.

### INTRODUCTION TO THE INVENTION

The present invention relates to modified release pharmaceutical compositions of particles coated with a valproic acid compound, which can be compressed into tablets or filled into capsules. The invention also relates to processes for preparing the compositions and their methods of use, treatment and administration. The present invention also relates to extended release pharmaceutical compositions of a valproic acid compound, wherein the compositions comprise a modified release polymer in a concentration greater than about 50% w/w of the total composition.

Divalproex sodium is a stable coordination compound having sodium valproate and valproic acid in a 1:1 molar ratio, formed by partially neutralizing valproic acid with sodium hydroxide. It has a chemical name sodium hydrogen bis(2-propylpentanoate), and structural Formula I, where n generally varies between about 2 and 6.

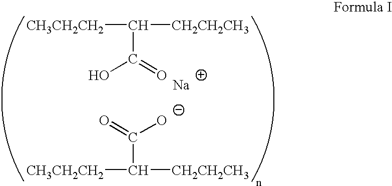

Divalproex sodium products are commercially available in three dosage forms for oral administration: DEPAKOTE® Sprinkle Capsules, containing coated particles of divalproex sodium equivalent to 125 mg of valproic acid in a hard gelatin capsule; DEPAKOTE® tablets that are enteric coated delayed release formulations having divalproex sodium equivalent to 125, 250, and 500 mg of valproic acid; and DEPAKOTE® ER (extended release) tablets that are hydroxypropyl methylcellulose matrix-based tablets having divalproex sodium equivalent to 250 and 500 mg of valproic acid. DEPAKOTE® sprinkle capsules are indicated for monotherapy and adjunctive therapy in the treatment of patients with various seizures, and the tablet forms are prescribed for treating seizure disorders, bipolar disorder, and migraine. DEPAKOTE® products are sold by Abbott Laboratories.

Divalproex sodium is effective in treating epilepsy, migraine, and mania in bipolar disorder. It dissociates within the gastrointestinal tract into valproate ion, which is responsible for eliciting the pharmacological response.

U.S. Pat. No. 6,610,326 discloses a delayed release dosage form containing divalproex sodium, wherein the divalproex sodium is not present in a 1:1 molar ratio of sodium valproate to valproic acid.

U.S. Pat. Nos. 5,212,326 and 4,988,731 disclose an oligomer having a 1:1 molar ratio of sodium valproate and valproic acid that shows superior stability characteristics as compared to valproic acid alone.

U.S. Pat. No. 5,169,642 discloses a sustained release dosage form in which the drug granules are coated with a sustained-release coating.

U.S. Pat. No. 4,913,906 discloses a controlled release oral dosage form comprising an essentially homogenous admixture of an salts and esters of valproic acid (VPA), valpromide, and other VPA derivatives in a concentration range of 10 to about 80 by weight, and a physiologically acceptable polymer.

U.S. Pat. No. 6,419,953 discloses a controlled release tablet dosage form comprising: a) a hydrophilic matrix formed from a uniform admixture of: i) about 50 weight percent to about 55 weight percent of valproic acid or its derivatives; ii) about 20 weight percent to about 40 weight percent of hydroxypropyl methylcellulose; and iii) about 5 weight percent to about 15 weight percent of lactose; b) from about 4 weight percent to about 6 weight percent of microcrystalline cellulose; and c) from about 1 about 5 weight percent of silicon dioxide;

U.S. Pat. No. 6,528,090 discloses an oral hydrophilic matrix formulation comprising: a) from about 40 to about 80% w/w of divalproex sodium; in admixture with b) a hydrophilic polymer in amounts from about 20% to about 50%, by weight of the formulation.

U.S. Pat. No. 6,713,086 discloses an oral polymeric controlled release formulation comprising: a) from about 40 to about 80% w/w of divalproex sodium; and b) a pharmaceutically acceptable hydrophilic polymer present in amounts from about 20% to about 50%, by weight of the formulation,

U.S. Patent Application Publication No. 2007/0160667 discloses a controlled release dosage formulation comprising: a) a valproic acid compound in an amount of about 40% to about 80% by weight of the dosage form; and b) at least two polymers, each in an amount less than about 20% of the tablet weight.

The widely used techniques for making a divalproex formulation are limited to granulation, subsequently coating the granules with a modified release coating and compressing them into tablets or filling them into capsules. The granulation techniques involves loss of drug during processing by way of generation of fines, since the fines generated do not pose a surface suitable for a modified release coating. An efficient modified release coating calls for a nucleus that has a specific surface area with a specific particle size. The nuclei generated by way of granulation have varying particle sizes with irregular and uneven surface area that leads to inefficient modified release coating. This subsequently results in erratic and inconsistent release profiles.

### SUMMARY OF THE INVENTION

Certain embodiments of the present invention relate to modified release pharmaceutical compositions of valproic acid compound coated particles, which can be compressed into tablets or filled into capsules without affecting the desired release profile. The invention further relates to processes for preparing the compositions of the invention, and their methods of use, treatment and administration.

In an aspect, the present includes a valproic acid compound composition that is prepared using a drug-layering technique, which comparatively incurs less drug loss during processing and results in substantially spherical particles. This enhances the efficiency of the subsequent modified release coating, thereby serving the purpose of achieving the desired release profile. Another aspect of the invention includes extended release tablets of a valproic acid compound comprising more than about 50% of a modified release agent, by weight of the total composition and achieving a desired drug release profile.

In an embodiment, the present invention includes modified release coated particle compositions of a valproic acid compound, comprising:
a) Pharmaceutically acceptable inert cores.
b) The inert cores optionally having a seal coating.
c) A valproic acid compound with or without pharmaceutically acceptable excipients layered or coated onto inert cores or onto seal coated inert cores.
d) The drug loaded particles optionally being coated with a modified release agent.

Aspects of the present invention further include modified release pharmaceutical compositions that comprise a valproic acid compound along with at least one modified release polymer, with or without other pharmaceutically acceptable excipients.

Aspects of the present invention further include modified release pharmaceutical compositions comprising a valproic acid compound and a hydrophilic matrix that is composed of at least more than about 50% hydrophilic polymer, by weight of the total composition.

In an embodiment, the invention comprises a pharmaceutical composition comprising a granulate comprising a valproic acid compound and either:

a) at least one diluent and less than about 5 percent by weight of a hydrophilic polymer; or

b) at least one hydrophilic polymer and less than about 5 weight percent of a diluent;

filled into a capsule, or compressed into a tablet having a coating that modifies release of a valproic acid compound from the tablet, wherein percentages are based on the weight of a capsule or tablet.

In another embodiment, the invention comprises a process to prepare a pharmaceutical composition comprising a valproic acid compound, comprising:

a) granulating a mixture comprising a valproic acid compound and one or more pharmaceutical excipients;

b) blending granules with at least one pharmaceutically acceptable excipient; and

c) compressing a blend of b) to form tablets and providing a coating that modifies release of a valproic acid compound from the tablets, or filling the blend into capsules.

In a further embodiment, the invention comprises a composition comprising a pharmacologically inert particle having a coating comprising a valproic acid compound, and an additional coating over the valproic acid compound coating that modifies release of a valproic acid compound from the composition.

In yet another embodiment, the invention comprises a process to prepare a pharmaceutical composition comprising modified release divalproex sodium-coated particles, comprising:

a) coating divalproex sodium, with or without a polymer, onto optionally seal coated pharmacologically inert particles;

b) optionally, coating the particles of a) with a coating that modifies release of divalproex sodium; and

compressing particles of a) or b) into tablets provided with a coating that modifies release of divalproex sodium from the tablets, or filling the particles into capsules.

### DETAILED DESCRIPTION

In an aspect, the present invention relates to modified release pharmaceutical compositions of valproic acid compound-coated particles which can be compressed into tablets or filled into capsules without affecting the desired release profile. The invention further relates to processes for preparing the compositions of the invention and their methods of use, treatment and administration.

Another aspect of the present invention includes modified release pharmaceutical compositions comprising a valproic acid compound along with at least one modified release polymer, with or without other pharmaceutically acceptable excipients.

Valproic acid compounds that are useful in the present invention include valproic acid and pharmaceutically acceptable salts, amides, esters, and prodrugs of valproic acid. Partial salts, such as divalproex sodium, are included within the scope of useful compounds.

Suitable pharmaceutically acceptable basic addition salts include, but are not limited to, valproic acid compounds having cations that are alkali metals, alkaline earth metals, and transition metals such as lithium, sodium, potassium, calcium, magnesium, aluminum, and the like, and ammonium and amine cations including ammonium, tetramethylammonium, tetraethylammonium, methylamine, dimethylamine, trimethylamile, triethylamine, diethylamine, ethylamine, and the like. Representative organic amines useful for the formation of base addition salts with valproic acid include ethylenediamine, ethanolamine, diethanolamine, piperidine, piperazine, and the like.

In accordance with the invention, pharmacologically inert cores comprise, without limitation thereto, pellets, beads, particles or nonpareil seeds that may be water-soluble or water-insoluble and organic or inorganic.

The inert cores generally have a particle size in the range of 100 to 850 μm, or about 150 to 300 μm, and comprise water-insoluble inert materials, such as glass particles or silicon dioxide, calcium phosphate dihydrate, dicalcium phosphate, calcium sulfate dihydrate, microcrystalline cellulose (including Celphere™ microcrystalline cellulose spheres sold by Asahi Kasei Chemicals Corporation, Tokyo, Japan), cellulose derivatives, or soluble inert materials such as sugar spheres having sugars like sucrose, dextrose, lactose, anhydrous lactose, spray-dried lactose, lactose monohydrate, or mannitol, starches, sorbitol, or insoluble inert plastic materials, i.e., spherical or nearly spherical core beads of polyvinylchloride, polystyrene or any other pharmaceutically acceptable insoluble synthetic polymeric material, and the like, and mixtures thereof.

In one embodiment of the present invention, the cores may be produced by any mechanical means, including extrusion spheronization, optionally using a binder during the granulation process.

In one aspect of the present invention, the cores have a defined size and shape, and enough strength to withstand fluidizing processes, thereby being resistant to attrition.

In one of the embodiments of the present invention, the cores are optionally seal coated to increase the strength of the core to withstand fluidizing processes. The seal coating helps to increase the mechanical strength of the cores by forming a tough insoluble coating on the surface and hence prevents material from shaving off during the fluidization process due to attrition. Useful seal coatings comprise pharmaceutically acceptable polymers, such as hydroxypropyl methylcellulose, ethyl cellulose, hydroxypropyl cellulose, methyl cellulose, carboxymethylcellulose, hydroxymethylcellulose, hydroxyethylcellulose, hydroxypropyl methyl phthalate, polyvinylpyrrolidone, cellulose acetate, waxes such as polyethylene glycol, methacrylic acid copolymers like poly(ethyl acrylate, methyl methacrylate, trimethylammonioethyl methacrylate chloride) (Eudragit™ RL), poly(ethyl acrylate, methyl methacrylate, trimethylammonioethyl methacrylate chloride) (Eudragit™ RS), and the like, and mixtures thereof.

In another embodiment of the present invention, the cores are coated with a first layer comprising of an active substance and subsequent layers comprising modified release polymer.

In an embodiment of the present invention, the active substance may be either dissolved or dispersed or suspended in a liquid, optionally containing a pharmaceutically acceptable binder, and may then be applied onto the cores.

In an embodiment of the present invention, the active substance may be either dissolved or dispersed or suspended in a solvent, optionally containing a pharmaceutically acceptable binder and a modified release polymer, and may then be applied onto the cores.

Examples of pharmaceutically acceptable binders include, but are not limited to, acacia, cellulose derivatives, gelatin, glucose, polyvinylpyrrolidone, sodium alginate and alginate derivatives, sorbitol, starch, hydrophilic cellulose excipients such as methylcellulose, hydroxypropyl methylcellulose and carboxymethylcellulose, xanthan gum, etc., and mixtures thereof.

In another embodiment of the present invention, a process to prepare the modified release divalproex sodium coated particle composition comprises:
1. Coating the active compound, dissolved or dispersed or suspended in a solvent system, with or without a polymer, onto cores by coating techniques such as powder coating, spray coating, dip coating, fluidized bed coating, and the like.
2. Optionally, coating the drug loaded cores with a modified release coating.
3. Compressing the drug loaded modified release particles into tablets or filling them into capsules.

In one embodiment of the present invention, the active compound is applied on the core using fluidized bed technology with Wurster or top spray or side spray techniques, from a dispersion or suspension or solution of the active compound, with or without a polymer, in a solvent system.

An aspect of the invention includes stable pharmaceutical compositions of a valproic acid compound, wherein a modified release coating provides stability to the composition by acting as a moisture barrier coating.

Solvents that can be used in processing include aqueous materials like water, alcohols like ethanol and isopropyl alcohol, hydro-alcoholic mixtures, organic solvents like acetone, methylene chloride and the like, and mixtures thereof.

When preparing the pharmaceutical preparation according to an embodiment of the invention it has surprisingly been found that the use of a solvent mixture of isopropyl alcohol and methylene chloride gives improved processability, by way of reducing agglomerate formation during drug loading. The ratio of isopropyl alcohol to methylene chloride may be varied from about 5:95 to 75:25 parts by volume, or about 15:85 to 30:75 parts by volume.

In one embodiment of the present invention, an optional seal coating may be applied between the drug loaded core and the modified release coating. The total amount of sealing layer contained in the drug-loaded particles may be varied depending on the desired release rate of the active compound.

In another embodiment of the present invention, drug-loaded cores are coated with a modified release polymer. Examples of suitable modified release polymers include but are not limited to ethyl cellulose, cellulose acetate, hydroxypropyl methylcellulose (different grades such as Methocel K 4M, Type 2208, Methocel E 4M Type 2910 supplied by Colorcon Asia Private Limited), hydroxypropyl cellulose, hydroxypropyl methyl pthalate, cellulose acetate pthalate, hydroxypropyl methylcellulose acetate succinate (HPMCAS), cellulose acetate trimellitate, Eudragit™ RL, Eudragit™ RS, polyvinylpyrrolidone, polyalkylene glycols such as polyethylene glycol, and cellulose derivatives such as hydroxypropyl cellulose, carboxymethyl cellulose, methyl cellulose, propyl cellulose, hydroxyethyl cellulose, carboxyethyl cellulose, carboxymethyl hydroxyethyl cellulose, hydroxymethylcellulose, carboxymethylethylcellulose, methyl-hydroxypropylcellulose, vinyl acetate copolymers, polysaccharides (such as alginate, xanthan gum and the like), polyethylene oxide, methacrylic acid copolymers, maleic anhydride/methyl vinyl ether copolymers and derivatives and mixtures thereof, acrylates such as methacrylates and acrylic acid copolymers; high molecular weight polyvinylalcohols; waxes like fatty acids and glycerides, and mixtures thereof.

Proprietary coating formulations like Acryl Eze®^{,} Surelease® and Kollidon SR® may also be used.

Plasticizers may be added to the polymeric solutions or suspensions in order to improve the processability or modify the drug release characteristics. Examples of plasticizers that may be used include but are not limited to citrate esters, acetylated monoglycerides, diacetylated monoglycerides, glycerine triacetate, triethyl citrate, acetyl triethyl citrate, tributyl citrate, polyethylene glycol, propylene glycol and mixtures thereof.

In addition to the above excipients, processing aids such as stearic acid or its derivatives may be used in the coating process to prevent agglomeration of the coating particles during the process of coating or after the coating.

In an embodiment of the present invention, the final dosage form comprises a large number of inert cores, which are layered with divalproex, dispersed or suspended or dissolved with a polymer in a solvent system.

In embodiments of the present invention, the final dosage form comprises a plurality of modified release particles and one or more pharmaceutically acceptable excipients compressed into a tablet. In other embodiments, a final dosage form of the present invention comprises a plurality of modified release particles filled into a hard gelatin capsule, with or without one or more pharmaceutically acceptable excipients. When coated particles are processed into tablets or capsules, the coated particles are further processed with any of the following described pharmaceutically acceptable excipients.

In another embodiment of the present invention, the final dosage form comprises an extended release composition comprising divalproex sodium mixed with at least one release modifying polymer, with or without one or more pharmaceutically acceptable excipients, compressed into a tablet, which optionally may be film coated.

In an embodiment, the invention includes extended release pharmaceutical compositions comprising divalproex sodium, wherein the modified release polymer is in a concentration range of more than about 50 percent by weight of the total composition.

The pharmaceutically acceptable excipients which may be used for further processing coated particles or which may be used to prepare the extended release compositions include the following.

### Diluents:

Various useful diluents include but are not limited to starches, lactose, mannitol, Pearlitol™ SD 200, cellulose derivatives, confectioners sugar and the like. Different grades of lactose include but are not limited to lactose monohydrate, lactose DT (direct tableting), lactose anhydrous, Flowlac™ (available from Meggle products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate.

### Binders:

Various useful binders include but are not limited to hydroxypropylcellulose (Klucel™-LF), hydroxypropyl methylcellulose or hypromellose (Methocel™), polyvinylpyrrolidone or povidone (PVP-K25, PVP-K29, PVP-K30, PVP-K90), plasdone S 630 (copovidone), powdered acacia, gelatin, guar gum, carbomer (e.g. carbopol), methylcellulose, polymethacrylates, and starch.

### Disintegrants:

Various useful disintegrants include but are not limited to carmellose calcium (Gotoku Yakuhin Co., Ltd.), carboxy methylstarch sodium (Matsutani Kagaku Co., Ltd., Kimura Sangyo Co., Ltd., etc.), croscarmellose sodium (FMC-Asahi Chemical Industry Co., Ltd.), crospovidone, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcellulose. Examples of low-substituted hydroxypropylcelluloses include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starch.

### Glidants:

Various glidants or antisticking agents include but are not limited to talc, silica derivatives, colloidal silicon dioxide and the like, and mixtures thereof.

### Lubricants:

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, zinc stearate, castor oils, and waxes.

Optionally, compressed tablets may be film coated using many of the same excipients as are used for seal coating. Several ready mixed coating materials are available, including those sold as OPADRY™ (supplied by Colorcon), for example Opadry grey 20A57646. These products require only mixing with a liquid, prior to application.

An opacifier like titianium dioxide may also be present in an amount ranging from about 10% (w/w) to about 20% (w/w) based on the total weight of the coating. Antiadhesives or antisticking agents are frequently used in the film coating process to avoid sticking effects during film formation and drying.

Suitable polishing agents include polyethylene glycols of various molecular weights or mixtures thereof, talc, surfactants (e.g. glycerol monostearate and poloxamers), fatty alcohols (e.g., stearyl alcohol, cetyl alcohol, lauryl alcohol and myristyl alcohol), waxes (e.g., carnauba wax, candelilla wax and white wax), acetylated monoglycerides, diacetylated monoglycerides, vanillin, etc.

In an embodiment, the invention includes compositions, which are formulated into solid oral dosage forms such as tablets, capsules, granules, etc.

In an embodiment the invention includes dosage forms, which are prepared by direct compression or dry granulation or wet granulation.

Equipment suitable for processing the pharmaceutical compositions of the present invention includes mechanical sifters, blenders, roller compacters, compression machines, rotating bowls or coating pans, fluid bed coaters with Wurster technology or top spray technology, etc.

In an embodiment, the invention includes processes to prepare extended release pharmaceutical compositions comprising divalproex sodium, comprising:
1 ) Sifting excipients through a sieve.
2) Mixing sifted excipients with the drug.
3) Optionally, roll compacting the drug and excipient mixture and subsequently milling and sifting the slugs.
4) Dissolving or dispersing a binder in a suitable solvent.
5) Granulating a dry mix of step 2 using the binder solution or dispersion of step 4.
6) Milling the wet granules through a suitable mill and subsequently drying.
7) Blending the dried granules from step 6 or from step 3 with sifted extragranular excipients.
8) Optionally, compressing the blend or alternatively filling into capsules.

The dosage forms prepared according to the present invention can be subjected to an in vitro dissolution evaluation, such as according to Test 711 “Dissolution” in United States Pharmacopoeia 24, United States Pharmacopeial Convention, Inc., Rockville, Md., 1999 (“USP”), to determine the rate at which the active substance is released from the dosage forms, and the content of active substance can be determined in solutions by high performance liquid chromatography. The pharmaceutical dosage forms of the present invention are intended for oral administration to a patient in need thereof.

In an embodiment, the invention includes the use of packaging materials such as containers and lids of high-density polyethylene (HDPE), low-density polyethylene (LDPE) and or polypropylene and/or glass, and blisters or strips composed of aluminum or high-density polypropylene, polyvinyl chloride, polyvinyl dichloride, etc.

The following examples illustrate certain specific aspects and embodiments of the invention and demonstrate the practice and advantages thereof. It is to be understood that the examples are given by way of illustration only and are not intended to limit the scope of the invention in any manner.

### EXAMPLE 1

### Composition of Divalproex 125 mg Capsules

| | |
|---|---|
| Ingredient | Kg/Batch |

SEAL COATED PARTICLES

| | |
|---|---|
| Sugar spheres (# 60/80 mesh) | 7 |
| Ethyl cellulose (7 cps) | 0.6 |
| Magnesium stearate | 0.6 |
| Triethyl citrate | 0.075 |
| Methylene chloride | 10.5 |
| Isopropyl alcohol | 6.45 |

DRUG LOADING

| | |
|---|---|
| Divalproex sodium | 31.782 |
| Hypromellose (5 cps) | 1.057 |
| Methylene chloride | 50.97 |
| Isopropyl alcohol | 10.55 |

MODIFIED RELEASE COATING

| | |
|---|---|
| Ethyl cellulose (7 cps) | 10.220 |
| Magnesium stearate | 10.22 |
| Triethyl citrate | 1.035 |
| Methylene chloride | 177.3 |
| Isopropyl alcohol | 108 |

### Excess quantities were taken in order to compensate for processing losses.

### Manufacturing Process:

A. Seal Coating:
1. Ethyl cellulose and triethyl citrate were dissolved in the mixture of isopropyl alcohol (3.45 Kg) and methylene chloride with stirring for 20 minutes to get a clear solution.
2. Magnesium stearate was dispersed in part of the isopropyl alcohol (3 Kg) and homogenized for 5 minutes using a colloid mill.
3. Step 2 dispersion was added to the step 1 solution and the stirring was continued for 5 minutes to obtain a homogenous dispersion.
4. Sugar spheres were loaded in a fluid bed coater and coated using the seal coating dispersion of step 3.

B. Drug Loading:
5. Hypromellose was dispersed in isopropyl alcohol using a mechanical stirrer.
6. Methylene chloride was added slowly to the dispersion and mixed for 2 minutes under stirring to obtain a clear solution.
7. Divalproex sodium was then added slowly to the solution and stirred until a homogeneous clear solution was obtained.
8. Seal coated sugar spheres of step 4 were loaded in a fluid bed coater and coated using the drug coating solution of step 7.

C. Modified Release Coating:
9. Ethyl cellulose and triethyl citrate were dissolved in the mixture of 63 Kg of isopropyl alcohol and methylene chloride with stirring.
10. Magnesium stearate was dispersed in the remaining 45 Kg of isopropyl alcohol and homogenized for 5 minutes.
11. Step 10 dispersion was added to the step 9 solution and the stirring was continued for 5 minutes to obtain a homogenous dispersion.
12. Drug loaded sugar spheres of step 8 were loaded into a fluid bed coater and coated using coating dispersion of step 11 until about 50% w/w coating build up was obtained.
13. The coated particles were then filled into size 1 hard gelatin capsules with a 250 mg average fill weight per capsule.

Coating Parameters:

| | Seal | Drug | Modified Release |
|---|---|---|---|
| Coating Liquid | Coating | Loading | Coating |
| Inlet air temperature (° C.) | 43 | 41 | 44 |
| Product temperature (° C.) | 34 | 34 | 34 |
| Solution flow rate | 72-112 | 50-115 | 100-200 |
| (grams/minute) | | | |
| Atomization pressure | 3.6 | 3.9 | 4 |
| (kg/cm²) | | | |
| Relative humidity (%) | 17 | 17 | 17 |

The in vitro release profile of the product of Example 1 in comparison with a commercial product was determined using the USP procedure with the following parameters:

Media: pH 7.5 phosphate buffer.

Volume: 500 ml.

Apparatus: USP apparatus II (Paddle).

Speed: 50 rpm.

Cumulative % Drug Dissolved

| Time (hours) | Depakote ® Sprinkle Capsules | Example 1 |
|---|---|---|
| 0 | 0 | 0 |
| 2 | 32 | 35 |
| 4 | 88 | 84 |
| 6 | 102 | 99 |
| 8 | 105 | 104 |
| 10 | 106 | 106 |

Bioavailability of the Example 1 product (“T”) was compared with the commercial product (“R”) using 34 human subjects in a 2-way crossover single dose fasted and fed state study, giving the following results:

| | Fasted | | Fed | |
|---|---|---|---|---|
| | | 90% | | 90% |
| | Mean T/R | Confidence | Mean T/R | Confidence |
| Parameter | Ratio | Interval | Ratio | Interval |
| AUC₀₋ₜ | 98.64 | 95.39-102 | 98.06 | 95.29-100.91 |
| AUC_{0-α} | 98.21 | 94.902-101.63 | 97.59 | 94.69-100.58 |
| Cₘₐₓ | 91.10 | 88.72-93.39 | 93.92 | 90.88-97.06 |

### EXAMPLE 2

### Composition of Divalproex 125 mg Capsules

| | |
|---|---|
| Ingredient | Quantity/Batch |

SEAL COATED PARTICLES

| | | |
|---|---|---|
| Sugar spheres (# 60/80 mesh) | 1000 | g |
| Ethyl cellulose (7 cps) | 47.63 | g |
| Magnesium stearate | 47.63 | g |
| Triethyl citrate | 4.74 | g |
| Methylene chloride | 500 | ml |
| Isopropyl alcohol | 500 | ml |

DRUG LOADING

| | | |
|---|---|---|
| Divalproex sodium | 1000 | g |
| Hypromellose (5 cps) | 30 | g |
| Methylene chloride | 800 | ml |
| Isopropyl alcohol | 200 | ml |

MODIFIED RELEASE COATING

| | | |
|---|---|---|
| Ethyl cellulose (7 cps) | 95.23 | g |
| Magnesium stearate | 95.23 | g |
| Triethyl citrate | 9.52 | g |
| Methylene chloride | 1000 | ml |
| Isopropyl alcohol | 1000 | ml |

Excess quantities were taken in order to compensate for processing losses. Manufacturing process: similar to that described in Example 1, with a capsule fill weight of 250 mg.

The in vitro release profile of the product of Example 2 in comparison with a commercial product was determined using the USP procedure with the following parameters:

Media: pH 1.2 (0.08N) HCl for 2 hours followed by pH 7.5 phosphate buffer.

Volume: 900 ml.

Apparatus: USP apparatus II (Paddle).

Speed: 50 rpm.

Cumulative % Drug Dissolved

| Time (minutes) | Depakote ® Sprinkle Capsules | Example 2 |
|---|---|---|
| 0 | 0 | 0 |
| 60 | 17 | 12 |
| 120 | 39 | 32 |
| 180 | 64 | 67 |
| 240 | 72 | 76 |
| 360 | 80 | 83 |
| 480 | 84 | 86 |

Bioavailability of the Example 2 product (“T”) was compared with the commercial product (“R”) using 14 human subjects in a 2-way crossover single dose fasted state study, giving the following results:

| Parameter | Mean T/R Ratio | 90% Confidence Interval |
|---|---|---|
| AUC₀₋ₜ | 106.503 | 100.3-113.11 |
| AUC_{0-α} | 105.675 | 100.19-111.45 |
| Cₘₐₓ | 99.75 | 95.34-104.37 |

### EXAMPLE 3

### Composition of Divalproex 125 mg Capsules Without Seal Coating

| | |
|---|---|
| Ingredient | Quantity/Batch |

DRUG LAYERING

| | | |
|---|---|---|
| Sugar spheres (# 60/80 mesh) | 200 | g |
| Divalproex sodium | 600 | g |
| Hypromellose phthalate | 30 | g |
| Colloidal silicon dioxide | 18 | g |
| Isopropyl alcohol | 910 | ml |
| Methylene chloride | 910 | ml |

MODIFIED RELEASE COATING

| | | |
|---|---|---|
| Ethyl cellulose (7 cps) | 47.62 | g |
| Magnesium stearate | 47.62 | g |
| Triethyl citrate | 4.76 | g |
| Isopropyl alcohol | 500 | ml |
| Magnesium stearate | 500 | ml |

DELAYED RELEASE COATING

| | | |
|---|---|---|
| Hypromellose phthalate (HP-55) | 104.35 | g |
| Triethyl citrate | 10.44 | g |
| Talc | 5.21 | g |
| Acetone | 360 | ml |
| Isopropyl alcohol | 840 | ml |

### Manufacturing Process:

A. Drug Layering:
1. Hypromellose and divalproex sodium were dissolved in a methylene chloride-isopropyl alcohol mixture, and then colloidal silicon dioxide was dispersed in the solution using a mechanical stirrer until a homogenous dispersion was obtained.
2. Drug solution of step 1 was coated onto sugar spheres using a fluid bed coating machine.

C. Modified Release Coating:
3. Ethyl cellulose and triethyl citrate were dissolved in the mixture of isopropyl alcohol and methylene chloride with stirring.
4. Magnesium stearate was dispersed in the step 3 solution until a homogenous dispersion was obtained.
5. Drug loaded particles of step 2 were loaded into a fluid bed coater and coated using coating dispersion of step 4 until about 20% w/w coating build up was obtained.

D. Delayed Release Coating:
6. Talc was dispersed in a mixture of isopropyl alcohol and acetone with mechanical stirring, then hypromellose phthalate and triethyl citrate were dissolved.
7. Particles of step 5 were loaded into a fluid bed coater and coated using coating dispersion of step 7 until about 20% w/w coating build up was obtained.
8. The coated particles were then filled into size 1 hard gelatin capsules with a 240 mg average fill weight per capsule.

### EXAMPLE 4

### Composition of Divalproex 125 mg Capsules With Different Particle Size Sugar Spheres

| | |
|---|---|
| Ingredient | Quantity/Batch |

DRUG LAYERING

| | | |
|---|---|---|
| Sugar spheres (# 30/35 mesh) | 300 | g |
| Divalproex sodium | 600 | g |
| Isopropyl alcohol | 200 | ml |
| Water | 200 | ml |

MODIFIED RELEASE COATING

| | | |
|---|---|---|
| Ethyl cellulose (7 cps) | 43.11 | g |
| Magnesium stearate | 43.11 | g |
| Triethyl citrate | 4.55 | g |
| Isopropyl alcohol | 450 | ml |
| Magnesium stearate | 450 | ml |

DELAYED RELEASE COATING

| | | |
|---|---|---|
| Hypromellose phthalate | 90.8 | g* |
| Triethyl citrate | 8.24 | g |
| Acetone | 225 | ml |
| Isopropyl alcohol | 675 | ml |

*10% excess taken to compensate for processing losses.

### Manufacturing Process:

A. Drug Layering:
1. Divalproex sodium was dissolved in a isopropyl alcohol-water mixture using a mechanical stirrer until a clear solution was obtained.
2. Divalproex sodium was loaded onto sugar spheres using the drug solution of step 1 using a fluid bed-coating machine.

B. Modified Release Coating:
3. Ethyl cellulose and triethyl citrate were dissolved in the mixture of isopropyl alcohol and methylene chloride with stirring.
4. Magnesium stearate was dispersed in the step 3 solution until a homogenous dispersion was obtained.
5. Drug loaded particles of step 2 were loaded into a fluid bed coater and coated using coating dispersion of step 4 until about 10% w/w coating build up was obtained.

C. Delayed Release Coating:
6. Hypromellose phthalate and triethyl citrate were dissolved in the mixture of isopropyl alcohol and acetone with stirring.
7. Particles of step 5 were loaded into a fluid bed coater and coated using coating dispersion of step 7 until about 10% w/w coating build up was obtained.
8. The coated particles were then filled into size 1 hard gelatin capsules with a 245 mg average fill weight per capsule.

### EXAMPLE 5

### Composition of Divalproex 125 mg Capsules

| | |
|---|---|
| Ingredient | Quantity/Batch |

DRY POWDER LAYERING

| | | |
|---|---|---|
| Microcrystalline cellulose | 300 | g |
| (Celphere ™ # 50/100 mesh) | | |
| Divalproex sodium | 900 | g |
| Hypromellose 5 cps | 30 | g |
| Isopropyl alcohol | 800 | ml |
| Water | 200 | ml |

MODIFIED RELEASE COATING

| | | |
|---|---|---|
| Ethyl cellulose (7 cps) | 28.93 | g |
| Magnesium stearate | 28.93 | g |
| Triethyl citrate | 2.91 | g |
| Methylene chloride | 305 | ml |
| Isopropyl alcohol | 305 | ml |

DELAYED RELEASE COATING

| | | |
|---|---|---|
| Hypromellose phthalate (HP-55) | 60.76 | g |
| Triethyl citrate | 6.08 | g |
| Acetone | 200 | ml |
| Isopropyl alcohol | 470 | ml |

### Manufacturing Process:

A. Drug Layering:
1. Hypromellose was dissolved in the isopropyl alcohol-water mixture using a mechanical stirrer.
2. Divalproex sodium was passed through a #20 mesh sieve.
3. Divalproex sodium was then loaded onto the Celphere #50/100 mesh using the binder solution of step 1 in a powder-layering machine.

B. Modified Release Coating:
4. Ethyl cellulose and triethyl citrate were dissolved in the mixture of isopropyl alcohol and methylene chloride with stirring.
5. Magnesium stearate was dispersed in the step 4 solution until a homogenous dispersion was obtained.
6. Drug loaded particles of step 3 were loaded into a fluid bed coater and coated using coating dispersion of step 5 until about 10% w/w coating build up was obtained.

C. Delayed Release Coating:
7. Hypromellose phthalate and triethyl citrate were dissolved in the mixture of isopropyl alcohol and acetone with stirring.
8. Particles of step 6 were loaded into a fluid bed coater and coated using coating solution of step 7 until about 10% w/w coating build up was obtained.
9. The coated particles were then filled into size 1 hard gelatin capsules with a 203 mg average fill weight per capsule.

### EXAMPLE 6

### Composition of Divalproex 500 mg Delayed Release Tablets

| Ingredient | Kg/Batch |
|---|---|
| Divalproex sodium | 69.95 |
| Microcrystallince cellulose (Avicel ™ PH112) | 22.35 |
| Pregelatinized starch (Starch 1500 LM) | 9.75 |
| Silicon dioxide (Syloid ™ 244 FP) | 2.6 |
| Povidone (PVP K-30) | 3.9 |
| Isopropyl alcohol | 16 |
| Pregelatinized starch (Starch 1500 LM) | 3.25 |
| Silicon dioxide (Syloid 244 FP) | 2.6 |
| Talc | 2.6 |
| Hypromellose 5 cps | 3.46 |
| Acetylated monoglycerides (Myvacet ™ 9-45)** | 0.052 |
| Isopropyl alcohol | 46 |
| Methylene chloride | 33.4 |
| Hypromellose phthalate (HP-55) | 7.07 |
| Acetylated monoglycerides (Myvacet ™ 9-45) | 1.056 |
| Titanium dioxde | 0.052 |
| Iron oxide (Ferric oxide red) | 0.007 |
| Acetone | 44.8 |
| Isopropyl alcohol | 19.4 |
| Acetylated monoglycerides (Myvacet ™ 9-45) | 0.07 |
| Vanillin | 0.13 |
| Isopropyl alcohol | 4.2 |
| Opacode Black S-1-8152HV* | 0.5 |
| Isopropyl alcohol | 5 |

*Opacode Black S-1-8152HV is an imprinting ink supplied by Colorcon (comprises iron oxide, shellac, soya lecithin and antifoam DC1510).
**Myvacet 9-45 is supplied by Kerry Bioscience, US.

### Manufacturing Process:

1. Divalproex sodium was deagglomerated in a rapid mixer granulator at high impeller and chopper speeds for about 20 minutes.

2. Microcrystalline cellulose (Avicel PH112), pregelatinized starch (Starch 1500 LM) and silicon dioxide (Syloid 244 FP) were sifted through a #40 ASTM mesh sieve.

3. Step 2 materials were added to step 1 and dry mixed at a high Impeller speed with the chopper off, for 15 minutes.

4. Povidone K30 was dissolved in isopropyl alcohol with stirring to obtain a clear solution.

5. Dry mixture of step 3 was granulated using the binder solution of step 4.

6. The wet granules were milled in a comminuting mill without any sieve with knife forward and medium speed.

7. The wet milled granules were dried in a fluid bed granulator at 50° C. until a LOD (loss on drying) was less than 1.5% w/w at 80° C.

8. Dried granules of step 7 were sifted through a #16 mesh sieve.

9. The extragranular excipients pregelatinized starch (Starch 1500 LM, silicon dioxide (Syloid 244 FP) and talc were passed through a ASTM 40# mesh sieve.

10. Loaded the granules of step 8 and ingredients of step 9 into a double cone blender, then blended for about 7 minutes.

11. Final blend was compressed to obtain tablets using 23×9.5 mm oval shaped plain/plain punches.

12. The tablets were coated using a coating solution (hypromellose 5 cps dissolved in isopropyl alcohol-methylene chloride (70:30) along with diacetylated monoglycerides) until a weight build up of about 2.5-3.5% w/w was obtained.

13. The barrier coated tablets were coated using the enteric coating dispersion (hypromellose phthalate dissolved/dispersed in isopropyl alcohol-acetone (65:35) along with diacetylated monoglycerides and pigments) until a weight build up of about 6-8% w/w, based on the tablet weight, was obtained.

14. The enteric-coated tablets were polished with the diacetylated monoglycerides and vanillin, dissolved in isopropyl alcohol.

15. The polished tablets were imprinted with identifying information using a tablet imprinting machine with Opacode Black S-1-8152HV. Final tablet weight was 991.5 mg.

### Dissolution Testing (USP Procedure):

Medium: 1 hour in (pH 1.2) 0.08N HCl, followed by 7.5 pH phosphate buffer.

Volume: 900 ml.

Apparatus: USP Type II (Paddle), 50 RPM.

Cumulative % Drug Dissolved

| Time (hours) | Depakote ® 500 mg | Example 6 |
|---|---|---|
| 0 | 0 | 0 |
| 10 | 2 | 3 |
| 20 | 39 | 29 |
| 30 | 85 | 64 |
| 45 | 103 | 89 |
| 60 | 104 | 93 |
| 80 | 104 | 96 |

### EXAMPLE 7

### Composition of Divalproex 500 mg Extended Release Tablets

| Ingredient | Kg/Batch |
|---|---|
| Divalproex sodium | 96.86 |
| Hypromellose (Methocel K4M, Type 2208) | 86.4 |
| Hypromellose (Methocel E4M, Type 2910) | 39.6 |
| Colloidal silicon dioxide (Aerosil 200) | 1.08 |
| Colloidal silicon dioxide (Aerosil 200) | 1.06 |
| Opadry grey 20A57646* | 2.25 |
| Hypromellose (Methocel E15LV premium) | 2.25 |
| Isopropyl alcohol | 73.8 |
| Methylene chloride | 53.5 |

*Opadry grey 20A57646 is supplied by Colorcon (composition comprises hydroxypropyl methylcellulose, hypromellose 6 cps/HPMC 2910, titanium dioxide, iron oxide black, and iron oxide yellow).

### Manufacturing Process:

1. Divalproex sodium was deagglomerated in a rapid mixer granulator by mixing at impeller and chopper high speed for about 20 minutes.

2. Hypromellose (Methocel K4M, Type 2208), hypromellose (Methocel E4M, Type 2910) and colloidal silicon dioxide (Aerosil 200, first quantity) were sifted together through a ASTM #30 mesh sieve in a mechanical sifter.

3. Step 2 and step 3 ingredients were mixed together in a rapid mixer granulator for about 20 minutes at impeller high speed and chopper off.

4. Dry mix of step 3 was roll compacted using a roller compactor to obtain slugs and the slugs were sifted through a ASTM #14 mesh sieve in a mechanical sifter.

5. The slugs were milled through a 4 mm screen using a comminuting mill with knives forward and slow speed. Sifted the milled slugs through a ASTM #40 mesh sieve. The material retained on a ASTM #40 mesh sieve was taken for further processing.

6. Colloidal silicon dioxide (Aerosil 200, second quantity) was sifted through a ASTM #16 mesh sieve. The sifted colloidal silicon dioxide was blended with the sifted and milled slugs of step 5 for 10 minutes in a double cone blender.

7. The final blend was compressed into tablets using 23×9.5 mm oval shaped plain/plain punches.

8. The tablets were coated using the Opadry and hypromellose dispersion prepared in isopropyl alcohol-methylene chloride (70:30) until a weight build up of about 1.5-2.5% w/w was obtained. Final tablet weight was 1275 mg.

### Dissolution Testing (USP Procedure):

Media: 0.1 N HCl, 500 ml for 45 minutes, followed by 0.05 M phosphate buffer+75 mM sodium lauryl sulfate (pH 5.5), 900 ml.

Apparatus: USP apparatus II (Paddle).

Speed: 100 rpm.

Cumulative % Drug Dissolved

| | Depakote ® ER, | |
|---|---|---|
| Time (hours) | 500 mg | Example 7 |
| 0 | 0 | 0 |
| 3 | 23 | 22 |
| 6 | 44 | 35 |
| 9 | 60 | 46 |
| 12 | 73 | 54 |
| 18 | 91 | 95 |
| 24 | 99 | 99 |

Bioavailability of the Example 7 product (“T”) was compared with a commercial product (“R”) using 34 human subjects in a 2-way crossover single dose fasted and fed state study, giving the following results:

| | Fasted | | Fed | |
|---|---|---|---|---|
| | | 90% | | 90% |
| | Mean T/R | Confidence | Mean T/R | Confidence |
| Parameter | Ratio | Interval | Ratio | Interval |
| AUC₀₋ₜ | 105.97 | 97.99-114.6 | 97.33 | 89.64-105.68 |
| AUC_{0-α} | 106.46 | 98.46-115.10 | 101.68 | 96.85-106.74 |
| Cₘₐₓ | 109.29 | 102.51-116.51 | 102.55 | 97.04-108.38 |

We claim:

1. A pharmaceutical composition comprising a granulate comprising a valproic acid compound and either:
a) at least one diluent and less than about 5 percent by weight of a hydrophilic polymer; or
b) at least one hydrophilic polymer and less than about 5 weight percent of a diluent;
filled into a capsule, or compressed into a tablet having a coating that modifies release of a valproic acid compound from the tablet, wherein percentages are based on the weight of a capsule or tablet.

2. The composition of claim 1, wherein a valproic acid compound, at least one diluent, and less than about 5 weight percent of a hydrophilic polymer are granulated.

3. The composition of claim 1, wherein a valproic acid compound, at least one hydrophilic polymer, and less than about 5 weight percent of a diluent are granulated.

4. The composition of claim 3, wherein hydrophilic polymer comprises at least about 50 percent by weight.

5. The composition of claim 3, wherein a hydrophilic polymer comprises hydroxypropyl methylcellulose.

6. The composition of claim 1, wherein a hydrophilic polymer comprises hydroxypropyl methylcellulose.

7. The composition of claim 1, wherein a valproic acid compound comprises divalproex sodium.

8. A process to prepare a pharmaceutical composition comprising a valproic acid compound, comprising:
a) granulating a mixture comprising a valproic acid compound and one or more pharmaceutical excipients;
b) blending granules with at least one pharmaceutically acceptable excipient; and
c) compressing a blend of b) to form tablets and providing a coating that modifies release of a valproic acid compound from the tablets, or filling the blend into capsules.

9. The process of claim 8, wherein in a) a valproic acid compound is coated onto a pharmacologically inert particle.

10. The process of claim 8, wherein in a) a mixture comprising a valproic acid compound is granulated together with a diluent, and a pharmaceutical composition comprises less than about 5 percent by weight of a hydrophilic polymer, based on the weight of a capsule or tablet.

11. The process of claim 10, wherein in a) a valproic acid compound is coated onto a pharmacologically inert particle.

12. The process of claim 10, wherein a valproic acid compound comprises divalproex sodium.

13. The process of claim 8, wherein in a) a mixture comprising valproic acid compound and a hydrophilic polymer is granulated, and a pharmaceutical composition comprises less than about 5 percent by weight of a diluent, based on the weight of a capsule or tablet.

14. The process of claim 13, wherein a hydrophilic polymer comprises hydroxypropyl methylcellulose.

15. The process of claim 13, wherein a valproic acid compound comprises divalproex sodium.

16. A composition comprising a pharmacologically inert particle having a coating comprising a valproic acid compound, and an additional coating over the valproic acid compound coating that modifies release of a valproic acid compound from the composition.

17. The composition of claim 16, wherein a pharmacologically inert particle has a seal coating, with a coating comprising a valproic acid compound being placed over a seal coating.

18. The composition of claim 16, wherein a pharmacologically inert particle is coated with a mixture of a valproic acid compound and a polymer.

19. The composition of claim 16, wherein a valproic acid compound comprises divalproex sodium.

20. A process to prepare a pharmaceutical composition comprising modified release divalproex sodium-coated particles, comprising:
a) coating divalproex sodium, with or without a polymer, onto optionally seal coated pharmacologically inert particles;
b) optionally, coating the particles of a) with a coating that modifies release of divalproex sodium; and
c) compressing particles of a) or b) into tablets provided with a coating that modifies release of divalproex sodium from the tablets, or filling the particles into capsules.
